(19) United States
(12) Patent Application Publication
(10) Pub. No.: US 2025/0391509 A1
(43) Pub. Date: Dec. 25, 2025

(54) SYSTEMS AND METHODS FOR ANALYTE DETECTION FROM MULTIPLEXED ASSAYS

(71) Applicant: LIFE TECHNOLOGIES CORPORATION, Carlsbad, CA (US)

(72) Inventors: Mark E. Shannon, San Francisco, CA (US); Carmen Gjerstad, Millbrae, CA (US); Harrison Leong, San Francisco, CA (US)

(73) Assignee: LIFE TECHNOLOGIES CORPORATION, Carlsbad, CA (US)

(21) Appl. No.: 18/879,442

(22) PCT Filed: Jun. 29, 2023
(86) PCT No.: PCT/US2023/069402

§ 371 Date: Dec. 27, 2024

Related U.S. Application Data
(60) Provisional application No. 63/356,863, filed on Jun. 29, 2022.
(60) Provisional application No. 63/356,874, filed on Jun. 29, 2022.
(60) Provisional application No. 63/408,665, filed on Sep. 21, 2022.

(51) Int. Cl. G16B 40/10; G01N 21/64

(52) U.S. Cl. CPC G16B 40/10; G01N 21/6428; G01N 2021/6439

(57) ABSTRACT

Systems and methods that enable analyte detection in a multiplexed amplification process can include obtaining, at multiple time points during the amplification process, composite emission signal data associated with a composite emission signal from at least a first probe type comprising a first label configured to generate a first emission signal and a second probe type comprising a second label configured to generate a second emission signal which has spectrally similar characteristics as said first emission signal. the first probe type and the second probe type differing in thermal and/or temporal properties; and determining, based at least partially on the composite emission signal data, emission signal data associated with a emission signal from a given probe type of the first probe type or the second probe type during the amplification process.

### CROSS-REFERENCE TO RELATED APPLICATIONS

This application claims priority to U.S. Provisional Application No. 63/356,874, titled “Systems and Methods for Enabling Multiplexed Polymerase Chain Reaction Processes” filed Jun. 29, 2022 U.S. Provisional Application No. 63/356,863, titled “Compositions and Methods for Detecting Nucleic Acids Using Intra-Channel Multiplexing” filed Jun. 29, 2022, and U.S. Provisional Application No. 63/408,665 titled “Compositions and Methods for Detecting Nucleic Acids Using Intra-Channel Multiplexing,” filed Sep. 21, 2022, the entire contents of each of which are incorporated herein by this reference.

### TECHNICAL FIELD

Aspects of the present disclosure relate to systems and methods for multiplex assay analyte detection. In particular, the present disclosure relates to nucleic acid detection using multiplex nucleic acid amplification assays, such as, for example polymerase chain reaction assay's.

### INTRODUCTION

Nucleic acid detection assays are often carried out by adding a sample that is suspected of including one or more target nucleic acids to a reaction mixture. The reaction mixture can include one or more detectable labels each designed to associate with a different target nucleic acid and generate a signal that corresponds to the amount of the associated target nucleic acid in the reaction mixture. In a “singleplex” assay, the reaction mixture includes a single detectable label designed to associate with a single target nucleic acid. Conversely, in a “multiplex” assay, the reaction mixture includes multiple, different detectable labels each typically designed to be specific to a different target nucleic acid.

Multiplex assays are therefore capable of detecting multiple different targets in a single reaction mixture. In some applications, the detectable labels are fluorescent dyes integrated with a nucleic acid probe, a primer, or some other nucleic acid molecule designed to specifically hybridize with the corresponding target nucleic acid with which it is designed to associate.

Challenges can arise when implementing multiplex systems and processes for determining the relative amounts of different target nucleic acids in a sample. In particular, using detectable labels that have spectral similarity (generate emission signal that are the same or overlap) can be challenging to determine the respective contributions of each label individually and thus of the respective different target nucleic acids with which they are associated.

A need exists to provide more robust systems and methods for carrying out multiplex nucleic acid detection assays, such as nucleic acid detection utilizing various polymerase chain reaction (PCR) assays for example, and for analyzing data associated with such assays.

### BRIEF DESCRIPTION OF THE DRAWINGS

Various objects, features, characteristics, and advantages of the inventions within the scope of the present disclosure will become apparent and more readily appreciated from the following description of various embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates emission spectra for various fluorescent dyes that can be used in nucleic acid detection assays;

FIG. 2A is a schematic overview of a technique for enabling detection of multiple target nucleic acids using detectable labels having spectral similarity, according to various embodiments of the present disclosure;

FIG. 2B is a graph showing signal response over time for the technique outlined in FIG. 2A, according to various embodiments of the present disclosure;

FIG. 3A illustrates activity of a cleavable probe and a non-cleavable probe during annealing, extension, and denaturation steps of a thermal cycle, according to embodiments of the present disclosure;

FIG. 3B is a graph showing fluorescent signal response over time during thermal cycling of an amplification process that utilizes the cleavable and non-cleavable probes of FIG. 3A, according to embodiments of the present disclosure;

FIG. 4 illustrates a flow diagram depicting exemplary actions associated with enabling analyte detection in a multiplexed amplification process, according to embodiments of the present disclosure;

FIGS. 5A and 5B illustrate results of a duplex assay test in which TaqMan probes and extendable fluorogenic (EF) probes were designed to generate spectrally similar fluorescence signals (FIG. 5A) or generate fluorescence signals of different spectral profiles (e.g., no significant overlap) (FIG. 5B);

FIG. 5C compares the EF-associated fluorescence signal as derived using the results of the assay of FIG. 5A with the EF-associated fluorescence signal as directly measured in the assay of FIG. 5B.

FIG. 5D illustrates fluorescent signals of a TaqMan probe and an extendable fluorogenic probe (EF) at extension and denaturation stages;

FIG. 6 illustrates the results of another 9-plex assay test that included 5 different detectable labels, with a corresponding TaqMan probe and an EF probe sharing four of the detectable labels and one of the detectable labels associated with only an TaqMan probe, according to embodiments of the present disclosure;

FIG. 7A illustrates a process of using a primer with a tail, specific to a nucleic acid target, to form a template to which the EF probe can hybridize; and

FIG. 7B illustrates an example forward primer with a tail, reverse primer, and EF probe that may be included in the reaction mixture to implement the process of FIG. 7A.

### DETAILED DESCRIPTION

In the context of a nucleic acid probe and a target nucleic acid, the term “specifically interact” (and similar terms) indicates that the probe is designed to interact with the target to a greater degree than with non-target nucleic acids also present in the reaction mixture. For example, specific interaction may include hybridization of the probe, in whole or in part, with the corresponding target. The hybridization between the probe and target need not be 100%. For example, functionally effective interaction may be accomplished with probes having homology to their respective target of at least about 70%, at least about 75%, at least about 80%, at least about 85%, at least about 90%, at least about 95%, at least about 97%, at least about 98%, at least about 99%, or up to 100%.

As used herein, a “detection channel” is a specified, subset of the total range of possible values of detectable signals. For example, where the detectable signals are fluorescence signals, a detection channel (e.g., fluorescence channel or dye channel) can represent a wavelength band of specified size. A detection channel may, for example, have a band size of about 10-60 nm, depending on instrument sensitivity and/or desired signal granularity. A detection channel can further include discontinuous wavelengths or wavelength ranges. A detection channel may additionally or alternatively be defined according to the optical filter arrangement used to measure the detectable signals. Each different detection channel typically comprises a specific optical filter arrangement to block non-channel emissions. Thus, as a functional definition, each detectable signal within a given optical filter arrangement may be considered as being within the same detection channel.

As used herein, separate fluorescence signals that have “substantially identical fluorescence” provide fluorescence emissions within similar wavelength bands. For example, a first fluorescence signal and a second fluorescence signal with substantially identical fluorescence may have emission peaks that differ by no more than about 10 nm, or no more than about 8 nm, or no more than about 6 nm, or no more than about 4 nm, or no more than about 2 nm, or no more than about 1 nm, or that are substantially indistinguishable from one another based on the sensitivity of the detection instrument used to measure the fluorescence emissions. Additionally, or alternatively, fluorescence signals may be considered to have “substantially identical fluorescence” in applications where they are measured using the same optical filter arrangement.

As used herein, a “substantial signal” and/or a detectable signal that has “substantial fluorescence” is a signal significantly above a background (i.e., baseline) level, including a fluorescence signal that is significantly above a background/baseline level of fluorescence. This may be defined by a threshold value that separates background fluorescence from substantial fluorescence. The threshold value may vary according to particular testing protocols and application needs. In some embodiments (without a passive reference), the threshold is set at about 1,000 to about 30,000, or more commonly about 2,000 to about 20,000, or about 3,000 to about 15,000 or about 4,000 to about 6,000, for example, or within a range having endpoints defined by any two of the foregoing values. In some embodiments (e.g., with a passive reference), the threshold is set at a change in fluorescence signal with the baseline detrended (ΔRn) of about 0.01 to 0.5, for example. In some embodiments, the threshold value is some percentage above the baseline level, such as about 5 percent to about 10 percent above the baseline level.

A “background” or “baseline” level of signal (i.e., background/baseline level of fluorescence) during an amplification process may be determined according to methods known to those of skill in the art. As a non-limiting example, the baseline level may be determined as the median signal of the amplification cycles before exponential amplification occurs. For example, exponential amplification may be determined when the change in signal from one amplification cycle to the next exceeds a certain percentage indicative of exponential change.

As a corollary, a signal and/or fluorescence level that is not “substantial” according to the foregoing may be described herein as “negligible.” Similarly, with respect to probe binding, a probe is “substantially bound” to its target when it is bound significantly above background (e.g., above binding to a non-target). Optionally, at least 1%, 5%, 10%, 20%, 50% or 80% of the probe or the target is bound.

As used herein, a “cleavable” probe is a probe that is intended to be cleaved as a result of specific interaction of the probe with its respective target, and to cause a release of the corresponding label and an increase in the corresponding detectable signal as a result.

As used herein, a “non-cleavable” probe is a probe with a label that is intended to remain associated with the probe throughout the assay. In a non-cleavable probe, the corresponding detectable signal varies according to configuration changes of the probe rather than by release of the label from the probe. An extendable fluorogenic probe is an example of a non-cleavable probe. For example, extendable fluorogenic probes can include universal or hairpin extendable fluorogenic probe, as described in various embodiments, or can have a structure of non-hairpin sequences.

The terms “detectable signal” and “label signal” are used synonymously herein. For example, a “first label signal” is the signal emitted by a first label of a first probe type and a “second label signal” is the signal emitted by a second label of a second probe type. A “total signal” is the total measured signal within a particular detection channel at a given time point or measurement point. Multiple different “detectable signals” /“label signals” may contribute to the same “total signal.” For example, a total signal may include signal generated by a first label of a first probe type and signal generated by a second label of a second probe type. In this regard, in some instances, a “total signal” may be regarded as a “composite signal.” In some embodiments, the signals are fluorescence signals, and terms such as “first fluorescence signal,” “second fluorescence signal,” and “total fluorescence signal” may be used as specific examples of the corresponding broader terms.

The term “spectral similarity” refers to the emission signal of detectable labels that have the same spectral profile or a substantially overlapping spectral profile. Thus, different probe types carrying the same detectable label or different probe types carrying different detectable labels with substantial spectral overlap in emission signal can both be considered probes with spectral similarity. In some implementations, detectable labels having spectral similarity can be detectable in a same optical detection channel, but other techniques can be used as well to detect the emission signals of such detectable labels. References made to substantially overlapping spectra should be understood to mean spectral similarity. While spectral similarity refers to emission signal exhibit the same or a degree of overlapping spectral signal and profile, the term does not refer to the time course or time periods over which such signal with the corresponding profile are emitted, such as during different stages of an amplification process.

The term “end-point” as referring to a cycle is a designated cycle at which the PCR process is assumed to be completed and/or a designated cycle at which a signal threshold that is above background signal by a defined amount occurs. In various embodiments, an endpoint cycle in accordance with the present disclosure may range from 20 to 45 cycles, for example, from 30-40 cycles. However, the number of cycles to an endpoint cycle may differ from these ranges and can be a designated cycle and/or a cycle correlated to where the emission (e.g., fluorescence) signal indicative of amplification product reaches a predefined level. And “end-point signal” refers to an emission signal measured during an end-point cycle.

The terms “determine,” “calculate,” and “estimate” are used synonymously herein. These terms are not intended to imply an exact level of measurement precision. Thus, where a value is “determined.” “calculated,” or “estimated” using the embodiments described herein, it will be understood that such a value may include some degree of inherent error due to factors such as detection instrument tolerances, rounding, chemical reaction variability, and other inherent measurement imperfections known and understood by those of skill in the art.

For any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about.” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

### Overview of Multiplexing Utilizing Probes With Detectable Labels Having Spectral Similarity

In various multiplex nucleic acid detection assays, each detectable label is assigned to a different target. The presence and/or amount of each target can then be determined by measuring the signal emitted from a detectable label, for example, in separate “detection channels” each corresponding to a specific property of the corresponding emitted signal. For example, in the context of fluorescence-emitting dyes as a detectable label, the separate detection channels can correspond to the emission wavelength spectrum associated with each dye. However, there can be some amount of overlap in the emission spectra of the different dyes. Increased overlap in emission spectra (spectral similarity) increases the difficulty in resolving the separate detected fluorescence emission signals and thus increases the difficulty in detecting and/or quantifying the respective targets.

While multiplexed dyes can be selected with the intent to minimize spectral overlap, the finite nature of the emission spectrum places practical limits on the number of separate dyes that can be combined in the same multiplex assay. As a result, at present, there are significant limitations to the number of different targets that can be detected and/or measured in a multiplex assay. Accordingly, there is an ongoing need for compositions and methods capable of increasing the “plexy” of detection assays. Moreover, it may be desirable to use detectable labels having spectral similarity, such as using dyes that have some degree of overlap in emission spectra and/or to use the same dye for different target nucleic acids.

Various embodiments disclosed herein pertain to systems and methods for enabling multiplexed nucleic acid detection assays that rely on polymerase chain reaction (PCR) processes by enabling determination of separate detectable signals, each associated with a different assay target nucleic acid, but that have spectral similarity. For example, various embodiments permit detection of spectrally similar detectable labels within the same detection channel (e.g., within a channel sensitive to emission (e.g, fluorescence emission) within a defined spectral range).

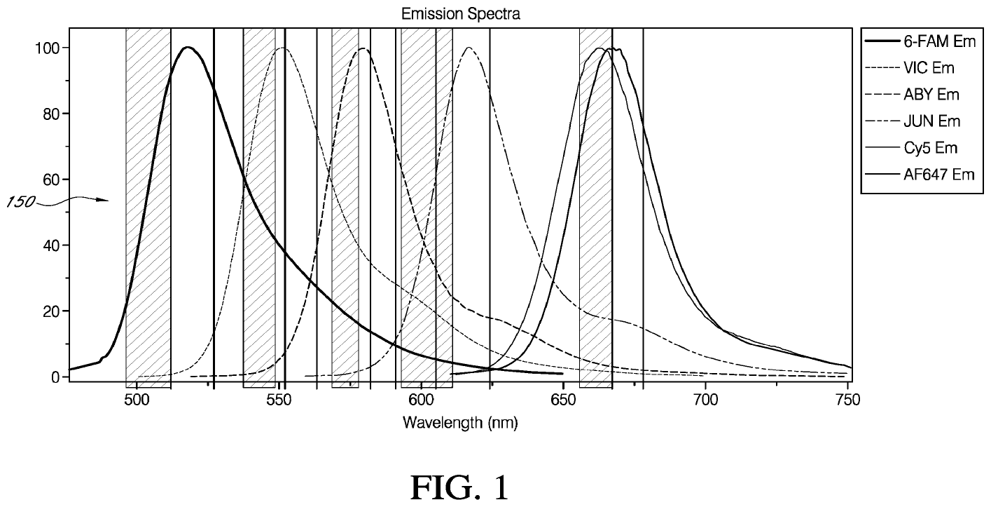

FIG. 1 illustrates emission spectra 150 for various fluorescent dyes 152 which can be used in nucleic acid detection assays. As discussed above, multiplex assays can assign each dye as a label for a separate target nucleic acid, and then determine the presence and/or amount of each target by measuring the fluorescence signal in separate detection channels that each correspond to differing emission wavelengths of the corresponding dye. As shown, there can be some (e.g., a substantial amount) of overlap in the emission spectra 150 of one or more of the dyes 152 such that those dyes are spectrally similar. For example, as illustrated in FIG. 1, the AF647 and Cy5 dyes have spectral similarity as they have substantially the same emission spectra. While multiplexed dyes are typically selected with the intent to minimize spectral overlap, the finite nature of the emission spectra places practical limits on the number of separate dyes that can be combined in the same multiplex assay, and therefore serves as a practical limit on the number of different targets that can be detected and/or measured without increasing a complexity of the system, such as increasing the number of separate detection channels and/or implementing other relatively complex deconvolution schemes.

Various embodiments described herein solve one or more of the foregoing problems by enabling analyte detection in multiplexed amplification processes by utilizing multiple detectable signals, each associated with a different assay target analyte or set of target analytes, that have spectral similarity in their emission spectra, and/or can be detected using the same detection channel. The multiple detectable signals can be separately resolved and independently analyzed to thereby allow detection and/or quantification of each target. By allowing multiple target analytes to be assayed utilizing the same detectable label (e.g., dye) or using detectable labels with spectral similarity, disclosed embodiments can beneficially increase the “plexy” (i.e., number of target analytes that can be detected and quantified in a multiplex assay) without relying on additional detectable labels (e.g., dyes), detection channels, and/or concomitant issues of spectral overlap. For example, a common detection channel can be used to detect labels having spectral similarity but that are intended for different target analytes in accordance with aspects of the present disclosure. Similarly, embodiments described herein can beneficially decrease the number of separate detectable labels (e.g., dyes) required in a multiplex assay without lowering the plexy of the assay. In addition, various embodiments can allow for the same detectable label (e.g. dye) to be used as a label for different target nucleic acids in a multiplex assay, including to use the same label for different target nucleic acids in a multiplex assay, including within a same cycle of a PCR reaction. Further, various embodiments can allow for detection of the same dye in a same detection channel.

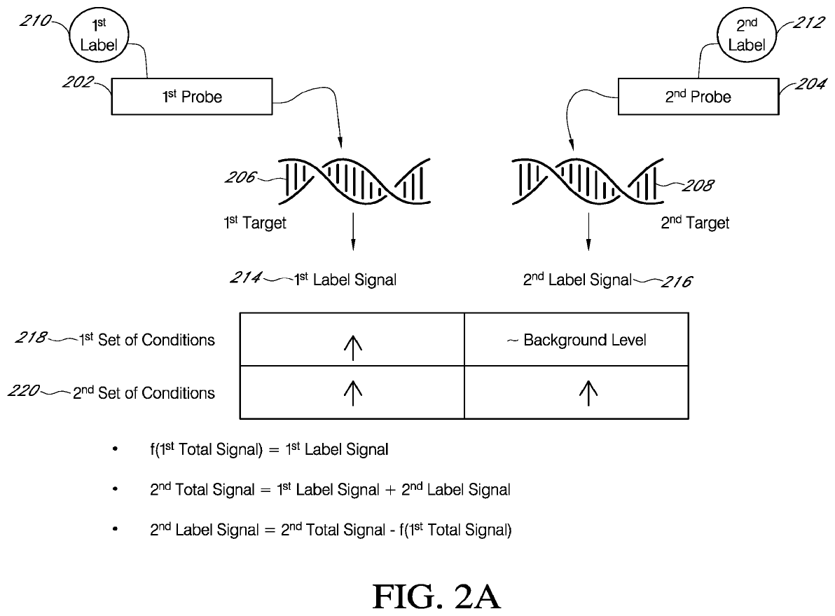

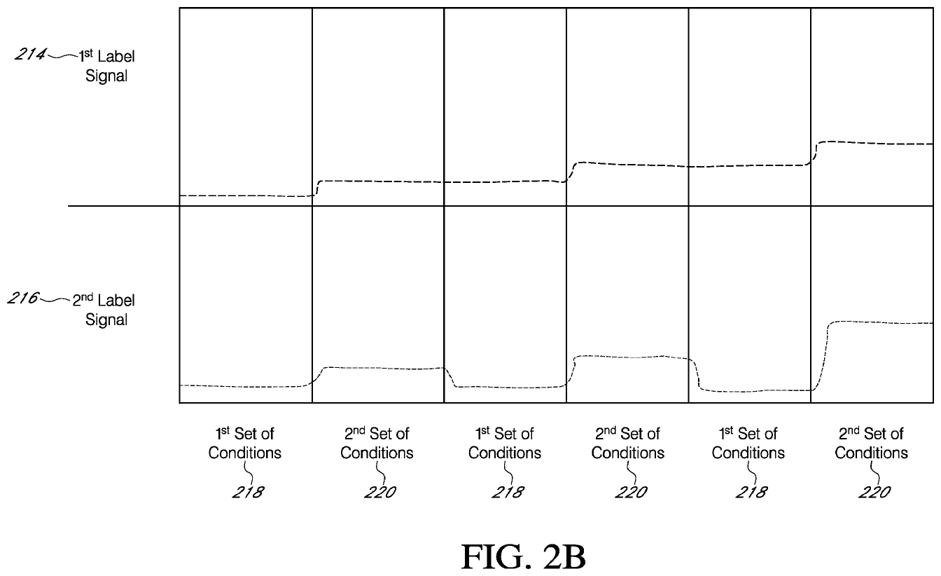

FIG. 2A is a schematic overview of a technique for enabling detection of multiple target nucleic acids utilizing detectable labels having spectral similarity by providing different first and second probe types, varying the reaction mixture conditions, and measuring the resulting total signal at each set of conditions. As shown, a first probe 202 is designed to specifically interact with a first target 206. The first probe 202 includes a first label 210 that can generate a first label signal 214. A second probe 204 is designed to specifically interact with a second target 208 that is different from the first target 206. The second probe 204 includes a second label 212 that can generate a second label signal 216.

In some embodiments, the first and second labels 210 and 212 are the same. For example, the first and second labels 210 and 212 may comprise the same fluorescent dye. In some embodiments, the first and second labels 210 and 212 may be different, but are nonetheless designed to generate a emission signals that have substantially identical or a degree of overlapping spectral profiles (e.g., have spectral similarity). For example, the first and second labels 210 and 212 may comprise dyes that are chemically distinct yet function to emit signals with similar wavelengths. In some embodiments, the first and second label signals 214 and 216 are measured using the same detection channel (e.g., including an optical filter arrangement) in the detection instrument.

The first probe 202 and second probe 204 may be provided in the same reaction mixture and allowed to specifically interact with any first and second target 206, 208, respectively, in the reaction mixture. As shown, the reaction mixture is subjected to at least two different sets of reaction conditions. The first probe 202 is designed such that the first label 210 generates the first label signal 214, to a degree proportional to the amount of specific interaction between the first probe 202 and first target 206, during both the first and second sets of conditions 218 and 220. In contrast, the second probe 204 is designed such that the second label 212 generates the second label signal 216, to a degree proportional to the amount of specific interaction between the second probe 204 and second target 208, during the second set of conditions 220 but not during the first set of conditions 218. In other words, under the first set of conditions 218, the first label signal 214 is increased as a result of specific interaction of the first probe 202 with the first target 206, but the second label signal 216 is not emitted as a result of specific interaction of the second probe 204 with the second target 208. Under the second set of conditions 220, the second label signal 216 is increased as a result of specific interaction of the second probe 204 with the second target 208, while the first label signal 214 also is further increased or remains at a relatively increased level to at least some degree from the first set of conditions.

During the first set of conditions 218, the second label 212 will not generate “substantial signal” (e.g., fluorescence) and the second label signal 216 will therefore not be substantially different from a background (i.e., baseline) level of emission signal (e.g., fluorescence) in the reaction mixture. That is, while there may be some non-zero level of signal generated by the second label 212 during the first set of conditions 218, the second label signal 216 will typically remain below a threshold value that separates background signal from meaningful signal. This threshold may vary according to particular testing protocols and application needs, as discussed above.

In at least some embodiments, when both the first and the second targets 206 and 208 are present in the reaction mixture, the second label signal 216 will differ between the first and second sets of conditions 218 and 220 to a greater degree than the first label signal 214 will differ between the first and second sets of conditions 218 and 220. Thus, while the first label signal 214 may differ somewhat between the first and second sets of conditions 218 and 220, this difference will typically be less than the difference in the second label signal 216 between the first and second sets of conditions 218 and 220.

Various embodiments of the present disclosure exploit the difference in the way the first and second label signals 214 and 216 respond to the different sets of conditions so as to enable the detected first and second label signals 214 and 216 to be resolved (separated), even, for example, if they are detected within the same detection channel (e.g., for a given optical filter arrangement that filters for a defined emission spectra in a channel). As illustrated, the total signal (or composite signal) during the first set of conditions 218 (“the first total signal”) is measured, and the total signal during the second set of conditions 220 (“the second total signal”) is measured. Fluorescence signal data representing the first total signal is sometimes referred to herein as “first fluorescence signal data”, and fluorescence signal data representing the second total signal is sometimes referred to herein as “second fluorescence signal data” or “composite fluorescence signal data”. As used herein, first and second in this context is not necessarily used to denote a temporal order of detection or the conditions, although such temporal order may occur.

During the first set of conditions 218, the total signal will be substantially equal to the first label signal 214. That is, the first total signal is primarily composed of the first label signal 214, whereas contribution from the second label signal 216 is negligible. During the second set of conditions 220, the total signal will include a combination of the first and second label signals 214 and 216. The first and second label signals 214 and 216 can therefore be separately resolved based on the first and second total signals. For example, the first label signal 214 can be determined based on the first total signal, and the second label signal 216 can be resolved by subtracting the first total signal from the second total signal.

In some embodiments, the first label signal 214 is equated directly to the first total signal. In other embodiments, the first label signal 214 is determined as a function of the first total signal. In some embodiments, this function is a linear function (though non-linear functions may be used in some implementations). For example, as discussed above, the first label signal 214 may differ slightly between the first and second sets of conditions 218 and 220 even when the amount of first target 206 has not changed. In certain applications, the first label signal 214 under the second set of conditions 220 may better correspond to standard curves that equate the first label signal 214 to first target 206 amounts. Estimating the first label signal 214 as a function of the first total signal, rather than as directly equal to the first total signal, can therefore bring the calculated first label signal 214 closer to what would be measured under the second set of conditions 220 (i.e., without any interfering second label signal 216).

In some embodiments, the function for converting the first total signal to the first label signal 214 is determined by comparing, in the absence of any second probe interacting with a second target, the first label signal 214 under the first set of conditions 218 to the first label signal 214 under the second set of conditions 220. The first label signal 214 under the first set of conditions 218 and under the second set of conditions 220 can be correlated to one another according to a linear function. In other embodiments, they can be correlated using non-linear functions. When a linear function is used, a multiplier factor (e.g., correction factor) can be used to convert the first total signal to the first label signal 214. Once such a linear function is determined, it can be used in subsequent assays without necessarily requiring additional comparisons of the first label signal 214 under the first set of conditions 218 and under the second set of conditions 220 in the absence of the second probe with the second target. As noted above, in some embodiments, the function for converting the first total signal to the first label signal may be non-linear. In some embodiments, the function/correlation is determined over stages of a thermal cycle or between thermal cycles at which the number of cleaved probes is expected to be the same. This approach can be used to resolve the different signals even if detected within the same detection channel, for example.

As described in greater detail below, the first probe 202 and the second probe 204 have different mechanisms of action that enable different signal responses, depending on the probe type, to the first and second sets of conditions 218 and 220. Beneficially, the ability to resolve the separate signals respectively associated with each of the different probe types can rely on attributes other than different melting temperatures of the probes. Thus, although the first probe 202 and second probe 204 may have dissimilar melting temperatures, such dissimilar melting temperatures is not a prerequisite to allow their associated label signals to be effectively resolved. In some embodiments, for example, a melting temperature (Tm) of the first probe 202 and a Tm of the second probe 204 are within about 8° C., or about 6° C. or about 4° C., or about 2° C. of each other, although such melting temperature differences are not limiting of the scope of the present disclosure. Moreover, in view of the techniques to enable differentiation in signal response in accordance with aspects of the present disclosure, a melting stage of an amplification process need not be relied on.

FIG. 2B is a graph schematically showing signal response over time for the technique outlined in FIG. 2A based on cycling of the reaction mixture between the first set of reaction conditions 218 and the second set of reaction conditions 220 and based on having both the first and second targets 206 and 208 present in the reaction mixture. The cycling of conditions may comprise, for example, the differing conditions of various stages associated with thermal cycling in a nucleic acid amplification reaction such as PCR for example. Under such a reaction, the first set of reaction conditions 218 correspond to supporting a denaturation stage of the thermal cycling and the second set of reaction conditions 220 correspond to supporting an annealing and/or extension stage (“annealing/extension stage”) of the thermal cycling. Thus, in various embodiments, the first set of reaction conditions 218 includes a first temperature or range of temperatures and the second set of reaction conditions includes a second temperature or range of temperatures (e.g., lower than the first).

As shown, both the first label signal 214 and the second label signal 216 increase under the second set of reaction conditions 220. Under the first set of reaction conditions 218, the first label signal 214 remains roughly the same as at the end of the previous cycle (though it may vary slightly, as discussed above), whereas the second label signal 216 drops to a level similar to the baseline signal level of the second label signal 216, which baseline signal level can be substantially constant over multiple amplification cycles. In other words, the second label signal 216 exhibits a baseline signal above a background signal level during the first set of reaction conditions. In some cases, the second label signal can exhibit a baseline signal level that changes at differing stages of an amplification cycle, but nevertheless is sufficiently distinguishable from and lower than the level under the second set of reaction conditions. This may be due to a different state of the probe and proximity of a quencher to the label.

As shown, both the first label signal 214 and the second label signal 216 cumulatively increase at each successive occurrence of the second set of conditions 220. This is a result of additional specific interaction in the reaction mixture between the first probe 202 and the first target 206 and additional specific interaction in the reaction mixture between the second probe 204 and the second target 208. However, where the first label signal 214 remains at a similar level when moving from the end of one cycle to the beginning of another (i.e., when moving from the second set of conditions 220 at the end of a cycle to the first set of conditions 218 at the beginning of a subsequent cycle), the second label signal 216 returns to a level near baseline at the beginning of each cycle (i.e., at each occurrence of the first set of conditions 218). In other words, the first label signal 214 is continuous and cumulative over subsequent cycles, the second signal is transient and dependent on the set of conditions occurring during a cycle.

While some embodiments described herein can be utilized with intra-channel multiplexing (detection of labels with a same detection channel), the disclosure is not limited to such. Moreover, the present disclosure contemplates intra-channel multiplexing being combined with inter-channel multiplexing to further increase the plexy of the assay. For example, an assay may be designed with multiple different detectable labels (e.g., dyes) having spectra for detection using multiple different detection channels, with one or more of the different channels configured to detect multiple detectable signals of signal responses from different targets using detectable signals that are spectrally similar and can be resolved in accordance with the techniques described herein. Moreover, by exploiting aspects of probes with detectable labels that are designed to interact and exhibit differing transitory emission signal patterns, it may be possible to use additional probe types along with the above-referenced probe types and take measurements at additional time periods over a reaction cycle so as to be able to further discern which signals correspond to which target nucleic acids.

### Cleavable & Non-Cleavable Probes

In some embodiments, the first probe (e.g., first probe 202) is a “cleavable” probe. The first probe may be designed such that the first label (e.g., first label 210) is detached from the first probe (and released from a corresponding quencher, for example) as a result of hybridization of the first probe to the first target (e.g., first target 206). Once released, the first label therefore continues to contribute to the total signal in the reaction mixture, thereby producing a cumulative emission signal. The first probe may be a TaqMan probe, for example, which undergoes cleavage as a result of 5′ to 3′ exonuclease activity of DNA polymerase during extension of the target molecule to which the probe is hybridized. TaqMan probes are described in U.S. Pat. Nos. 4,889,818; 5,079,352; 5,210,015; 5,436,134; 5,487,972; 5,658,751; 5,210,015; 5,487,972; 5,538,848; 5,618,711; 5,677,152; 5,723,591; 5,773,258; 5,789,224; 5,801,155; 5,804,375; 5,876,930; 5,994,056; 6,030,787; 6,084,102; 6,127,155; 6,171,785; 6,214,979; 6,258,569; 6,814,934; 6,821,727; 7,141,377; and 7,445,900, all of which are hereby incorporated herein by reference.

In some embodiments, the second probe (e.g., second probe 204) is a “non-cleavable” probe. The label of a non-cleavable probe is intended to remain associated with the probe throughout the assay, and to vary in the level of generated signal according to probe configuration rather than release of the label. The second probe may be an extendable fluorogenic probe (EF), for example, which quenches the label when in a single-stranded configuration but allows signal when incorporated into a double-stranded molecule (i.e., producing a transitory emission signal).

EF probes can be, for example, a universal extendable fluorogenic probe, an extendable hairpin probe designed for specific target amplification, or an extendable probe with a structure of non-hairpin sequences.

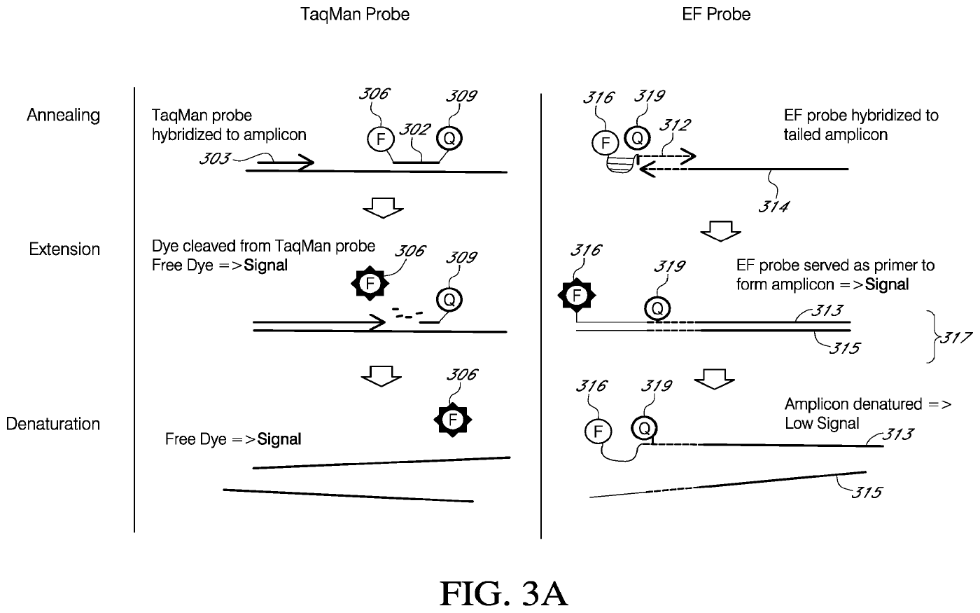

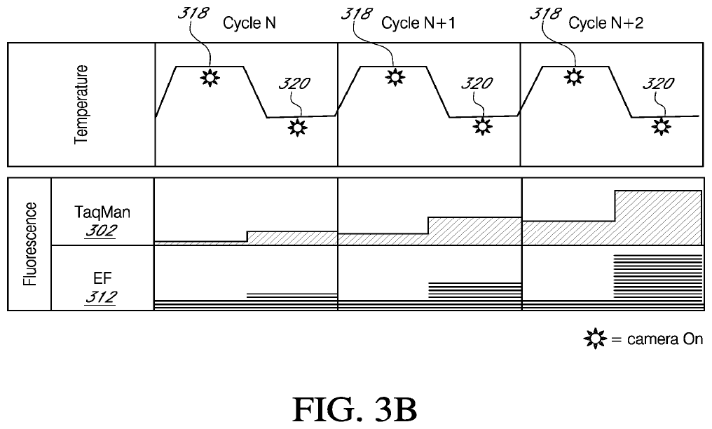

FIG. 3A illustrates activity of a cleavable probe 302, which in various embodiments can be a TaqMan probe, and a non-cleavable probe 312, which in various embodiments can be a EF probe, during annealing, extension, and denaturation stages of a PCR reaction thermal cycle. As shown, the TaqMan probe 302 hybridizes to its corresponding target nucleic acid amplicon 304 (as used herein target nucleic acid amplicon can refer to a single strand of the target double-stranded nucleic acid and should be understood by reference to the context when describing a PCR reaction) during the annealing stage. During extension of a primer 303 hybridized to the target nucleic acid amplicon 304 upstream of the probe 302, the 5′ to 3′ exonuclease activity of a DNA polymerase cleaves the TaqMan probe label 306 from the remainder of the probe 302, thereby separating it from the corresponding TaqMan probe quencher 309. This leads to a corresponding increase in the fluorescence signal. During denaturation, the label 306 remains free within the reaction mixture solution and thus continues to contribute to the total fluorescence signal.

The EF 312 includes a EF label 316 and a EF quencher 319 which remain in proximity to one another while the extendable probe 312 is in a single-stranded configuration. The fluorescence signal from the label 316 thus remains substantially quenched while the EF is in a single-stranded configuration. During the annealing and extension stages, the EF 312 hybridizes to its corresponding target template amplicon 314 and is extended to form an extended probe amplicon 313. Extension of target template 314 then forms the complement 315 of the extended probe amplicon 313. The resulting double-stranded amplicon 317 forces the label 316 away from the quencher 31 to a distance sufficient to allow fluorescence emission. During denaturation, the extended probe amplicon 313 is separated from its complement 315. When returned to the single-stranded configuration, the label 316 and quencher 319 are brought back into proximity and fluorescence is again quenched.

FIG. 3B is a graph showing the fluorescence signals from the TaqMan probes 302 and the EF probes 312 over time during thermal cycling of an amplification process. The temperatures of the thermal cycling may be varied according to particular application needs. As an example, the denaturation stage may be carried out at a temperature in a range of from about 80° C. to about 100° C., for example, from about 85° C. to about 95° C., for example, from about 90° C. to about 95° C. The annealing/extension stage may be carried out at a lower temperature, such as in a range from about 40° C. to about 75° C., for example from about 50° C. to about 70° C., for example from about 55° C. to about 65° C. In some implementations, the first set of reaction conditions (e.g., first set of conditions 218, as discussed with reference to FIG. 2A) corresponds to a denaturation stage 318, while the second set of reaction conditions (e.g., second set of conditions 220, as discussed with reference to FIG. 2A) corresponds to an annealing/extension stage 320.

While various embodiments cycle between a denaturation stage 318 and a combined annealing/extension stage 320 (i.e., the amplification process cycles between two target temperatures), other embodiments may include separate annealing and extension stages. In such embodiments, the temperature, and possibly other reaction conditions, may be varied between the annealing and the extension stages. For example, the extension stage can be carried out at a higher temperature than the annealing stage temperature. In some embodiments, the amplification process cycles between two differing target temperatures or two differing target temperature ranges for at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the cycles of the amplification process.

FIG. 3B shows that the fluorescence signal associated with the TaqMan probe 302 increases during the extension stage 320 and then remains at a similar level through the denaturation stage 318 of the next cycle (although some relatively insignificant decrease in signal can occur as described above), whereas the fluorescence signal associated with the EF probe 304 increases during the extension stage 320 but decreases to the baseline signal level associated with the EF probe 304 once the subsequent denaturation stage 318 reaches the target denaturation temperature. Those having ordinary skill in the art would appreciate that the cycles N, N+1, N+2 of FIG. 3B may begin at a different stage, however, in which case the comparison of signal levels noted above may be shifted.

In some embodiments, the first set of reaction conditions (e.g., the denaturation conditions 318) includes a first measurement temperature at which the first label signal is measured, and the second set of reaction conditions (e.g., the annealing/extension conditions 320) includes a second, different measurement temperature at which the first and second label signal is measured. In some embodiments, the first and second measurement temperatures differ by at least about 10° C. or more, about 15° C. or more, about 20° C. or more, about 25° C. or more, or about 30° C. or more. The first measurement temperature may be the target denaturation temperature in a range of, for example, about 80° C. to about 100° C., for example, from about 85° C. to about 95° C., for example, from about 90° C. to about 95° C., and the second measurement temperature may be the target annealing/extension temperature in a range of, for example, from about 40° C. to about 75° C., for example, from about 50° C. to about 70° C., for example from about 55° C. to about 65° C.

### Example Techniques and Implementations

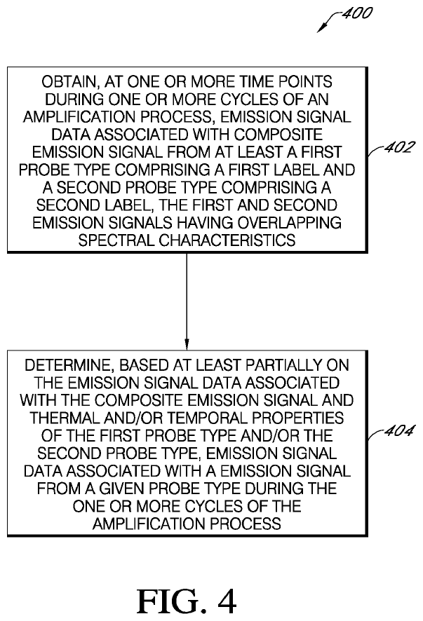

Disclosed techniques for enabling analyte detection in multiplexed amplification processes may entail the performance of various acts. For instance, FIG. 4 illustrates an example flow diagram 400 depicting acts associated with enabling analyte detection in a multiplexed amplification process (e.g., a multiplexed polymerase chain reaction (PCR) process).

Act 402 of flow diagram 400 includes obtaining, at one or more time points during one or more cycles of an amplification process, emission (e.g., fluorescence) signal data associated with a composite emission (e.g., fluorescence) signal from at least a first probe type comprising a first detectable label (e.g., fluorophore) and a second probe type comprising a second detectable label (e.g., fluorophore) which has spectral characteristics that are spectrally similar to the first detectable label. The first probe type and the second probe type may be selected/configured such that their associated label signals respond differently to different sets of reaction conditions (e.g., similar to the first probe 202 and second probe 204 discussed hereinabove with reference to FIG. 2A). For instance, the first probe type and the second probe type may differ in thermal and/or temporal properties. As noted above, the first and second detectable labels can have spectral similarity, such that both may be configured to generate emission signal (e.g., fluorescence) with a substantially similar range of wavelengths (e.g., corresponding to the first label and the second label 212 discussed hereinabove with reference to FIG. 2A).

A composite signal may comprise an emission (e.g., fluorescence signal) that both the first probe type and second probe type substantially contribute to. As noted above, emission (e.g., fluorescence) signal data associated with such a composite signal (and captured during particular reaction conditions, such as particular temperature conditions) may be regarded as “composite emission signal data” or “second emission signal data” or “composite fluorescence signal data” or “second fluorescence signal data.”

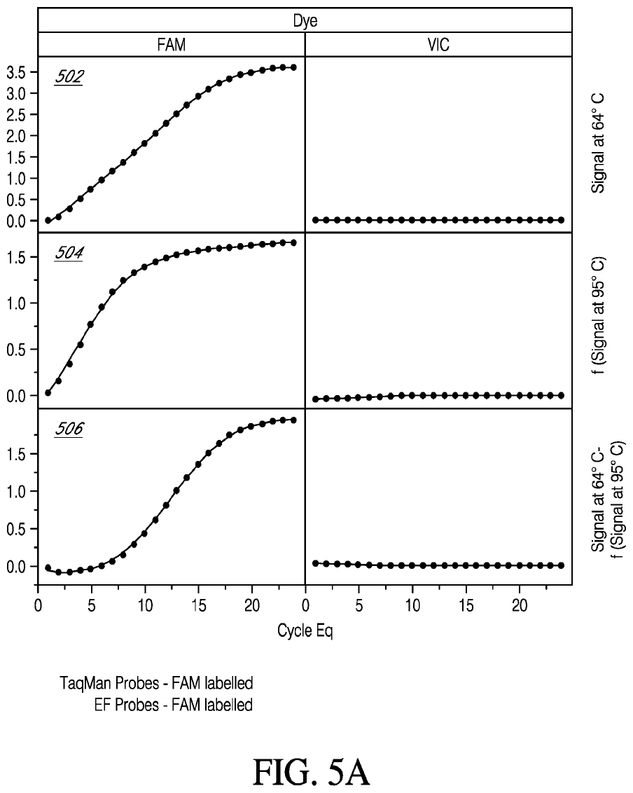

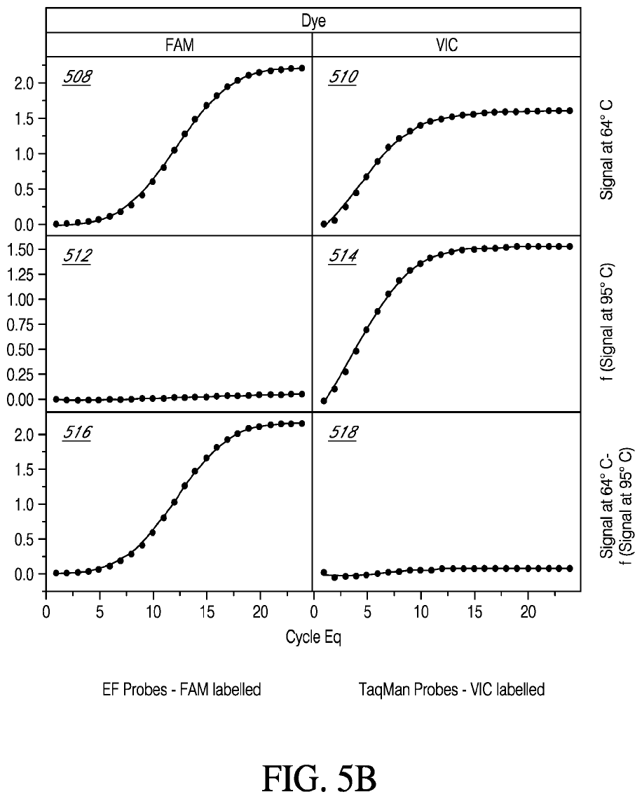

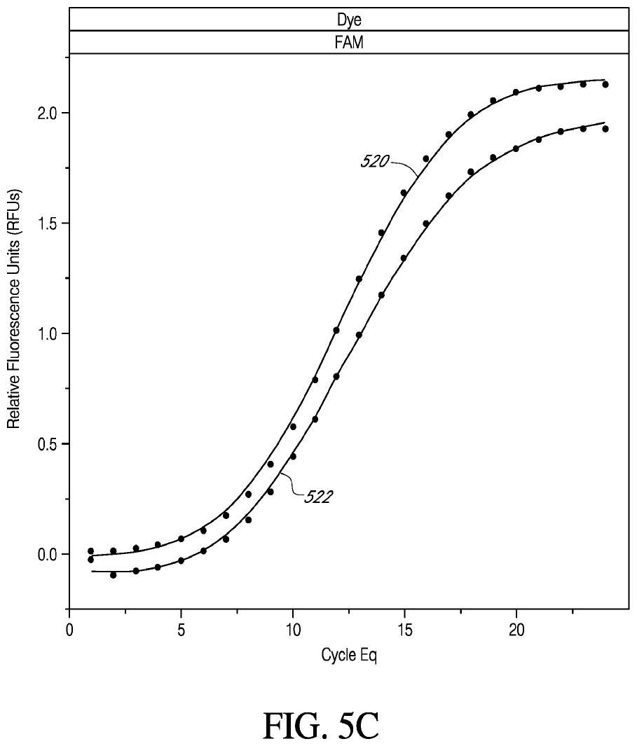

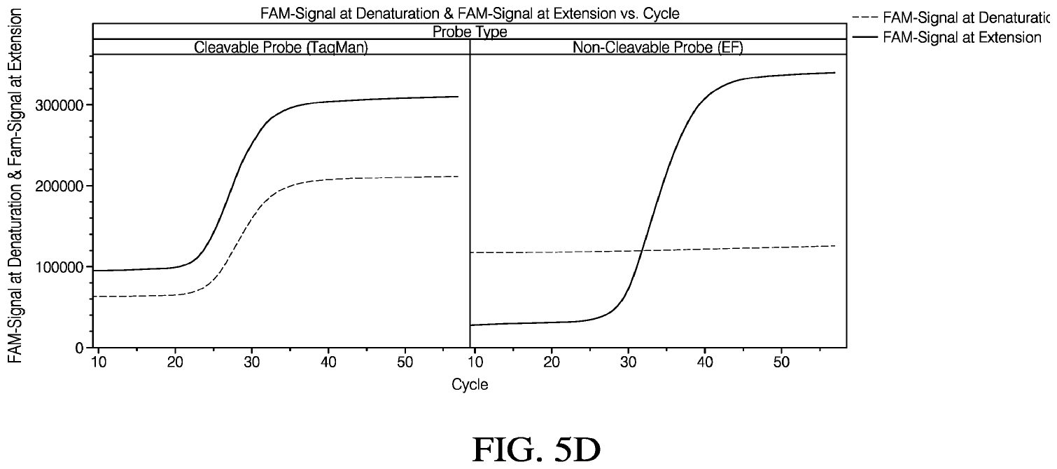

As will be discussed in more detail hereinafter, FIG. 5A provides an example graphical depiction of composite fluorescence signal data from a first probe type and a second probe type (e.g., where TaqMan and EF probes are labeled with FAM fluorophore, the FAM signal at 64° C. (top row) representing a composite fluorescence signal). In some instances, the first fluorophore of the first probe type and the second fluorophore of the second probe type are the same (e.g., in FIG. 5A where both the TaqMan and EF probes are FAM labelled). Notwithstanding the potential commonality between the fluorophores of the two probe types, the two probe types can be otherwise configured to have respective binding affinity to different analytes (e.g., different target nucleic acids). For example, the first probe type may have binding affinity to a first analyte (e.g., corresponding to the first probe 202 being designed to interact with the first target 206, as discussed above with reference to FIG. 2A) and the second probe type may have binding affinity to a second analyte that is different from the first analyte (e.g., corresponding to the second probe 204 being designed to interact with the second target 208, as discussed above with reference to FIG. 2A).

In some implementations, the one probe type (e.g., the first probe type) is a cleavable probe and the other probe type(s) (e.g., the second probe type) is/are non-cleavable. In some implementations, the cleavable probe type is a TaqMan probe, and the non-cleavable probe type is an EF probe (e.g., in the examples discussed with reference to FIGS. 3A, 3B, and 5A through 5D). The first probe type may be associated with a cumulative fluorescence that stays substantially stable across multiple segments of an amplification cycle and increases cumulatively over multiple amplification cycles, whereas the second probe type may be associated with a transient fluorescence that fluctuates significantly over each amplification cycle, such as during differing stages of an amplification cycle.

Act 404 of flow diagram 400 includes determining, based at least partially on the emission (e.g., fluorescence) signal data associated with the composite emission signal and thermal and/or temporal properties of the first probe type and/or the second probe type, emission (e.g., fluorescence) signal data associated with an emission (e.g., fluorescence) signal from a given probe type during the one or more cycles of the amplification process. The given probe type with which the determined emission (e.g., fluorescence) signal is associated may comprise at least one of the first probe type or the second probe type (e.g., the derived EF-associated signal of FIG. 5C).

In some implementations, first emission signal data is acquired at multiple timepoints during the amplification process from which the composite emission signal data is obtained. The first emission signal data may be associated with different reaction conditions than reaction conditions associated with the composite emission signal data. For instance, the composite emission signal data may be associated with a second set of reaction conditions (e.g., corresponding to the second set of conditions 220, 320 discussed above with reference to FIGS. 2A, 2B, and 3B) and the first emission signal data may be associated with a first set of reaction conditions (e.g., corresponding to the first set of conditions 218, 318 discussed above with reference to FIGS. 2A, 2B, and 3B).

In some instances, the different sets of reaction conditions are associated with different temperature conditions, such as different temperatures or different ranges of temperatures (e.g., a first temperature condition associated with the first emission signal data, and a second temperature condition associated with the second or composite emission signal data). In some instances, the first temperature condition is higher than and/or does not overlap with the second temperature condition. For example. FIG. 3B depicts a second temperature condition of 65° C., which may be associated with the composite emission signal data, and a first temperature condition of 95° C., which may be associated with the second emission signal data. Other temperature conditions are within the scope of the present disclosure. (e.g., a second temperature condition within a range of about 40°-75° C.; a first temperature condition within a range of about 80°-100° C.). The first and second temperature conditions can be associated with different stages of the amplification process. For example, the second temperature condition may be associated with an annealing stage or extension stage of the amplification process or a combined extension/annealing stage (e.g., second set of conditions 320 of the various amplification cycles represented in FIG. 3B), whereas the first temperature condition may be associated with a denaturing stage of the amplification process (e.g., first set of conditions 318 of the various amplification cycles represented in FIG. 3B).

In view of the reaction difference in reaction conditions, the composite and first emission signal data may accordingly be associated with different timepoints or time periods within an amplification process. For example, datapoints (or subsets of datapoints) that form the composite emission signal and the first emission signal data may be acquired/captured in a temporally interleaved manner (e.g., alternating between capturing datapoints for the composite emission signal data and the first emission signal data as the reaction conditions alternate during the amplification process). Further, the first emission signal and composite emission signal can be captured by collecting several data points within the course of an amplification (e.g., PCR) cycle and separating the two signals according to their differing time characteristics/profiles. As noted above, this technique can also be utilized with more than two probe types with detectable labels by leveraging the different temporal aspects of the various emission signals.

Determining the emission signal data associated with the emission signal for the given probe type may utilize as inputs the composite emission signal data (discussed above with reference to act 402) and the first emission signal data, as will be described in more detail hereinbelow with reference to FIG. 5A (e.g., referring briefly to FIG. 5A, the signal data in the top row, left column represents example measured composite fluorescence signal data; the signal data in the middle row, left column represents example transformed first fluorescence signal data determined using measured first fluorescence signal data; and the signal data in the bottom row, left column represents example fluorescence signal data associated with the fluorescence signal for the given probe). The fluorescence signal data associated with the fluorescence signal for the given probe may be determined in real-time during the amplification process, or as a post-processing operation.

FIGS. 5A and 5B illustrate results of a qPCR duplex assay in which TaqMan and EF probes were designed to generate fluorescence signals having spectral similarity, and for example, detectable in the same dye channel (FIG. 5A) or to generate different fluorescence signals without significant overlapping spectra, and for example, detectable in different dye channels (FIG. 5B). In the assay shown in FIG. 5A, both the TaqMan probes and the EF probes were labelled with FAM, and the VIC fluorophore signal (depicted in the right handle column of data results in FIG. 5A) thus served as a control. In the assay shown in FIG. 5B, the TaqMan probes were labeled with VIC and the EFs were labelled with FAM. The reaction mixture composition, template DNA concentrations, and amplification conditions were otherwise held the same in the two assays.

In FIG. 5A, graph 502 shows in the column on the left the change in FAM fluorescence signal with the baseline signal detrended (ΔRn) (y-axis) over cycle number (x-axis) measured at a temperature during the annealing/extension stage (64° C. in this example). This signal is expected to include fluorescence generated by both the TaqMan probe labels (those that have been cleaved from the probes) and the EF probe labels (those that have been incorporated into double-stranded amplicons). The FAM signal of graph 502 of FIG. 5A may comprise composite fluorescence signal data, as discussed above with reference to FIG. 4.

Graph 504 of FIG. 5A shows the change in FAM fluorescence signal with the baseline detrended (ΔRn) (over cycle number measured at a temperature during the denaturation stage (95° C. in this example) and modified by a linear function that correlates the 95° C. measurement to a 64° C. measurement for the TaqMan probes. This signal indicates an approximate fluorescence associated with the TaqMan probes at 95° C. and is expected to include fluorescence generated by the TaqMan probe labels but not to include significant fluorescence from the EF probe labels. The FAM fluorescence signal data measured at 95° C. and linearly transformed to provide the signal data shown in graph 504 of FIG. 5A may comprise “first fluorescence signal data” as discussed as above with reference to FIG. 4. The transformed fluorescence signal data depicted in graph 504 of FIG. 5A may thus be regarded as “transformed first fluorescence data.” As noted above, the function used to correlate the 95° C. measurement to the 64° C. measurement may be determined by comparing TaqMan probe signals measured according to the different temperature conditions (e.g., in the absence of EF or other probe types of spectral similarity, e.g., in the same spectral channel).

In the example of FIG. 5A, the composite fluorescence signal data (e.g., in graph 502 of FIG. 5A) is measured according to a second set of reaction conditions (i.e., at an annealing or extension stage temperature (or combined annealing/extension stages of 64° C.) and captures fluorescence signals from both probe types (i.e., TaqMan and EF probes). The transformed first fluorescence signal data (e.g., of graph 504 of FIG. 5A) approximates the fluorescence signal from one of the probe types (i.e., TaqMan probe type) according to the first set of reaction conditions (i.e., at the denaturation stage temperature of 95° C.). As noted above, the composite fluorescence signal data and the transformed first fluorescence signal data may be used to determine the fluorescence signal from the other probe type (EF probe type) according to the second set of reaction conditions (i.e., at the annealing or extension temperature of 64° C.). The composite fluorescence signal data may be modified by the transformed first fluorescence signal data (e.g., by subtracting the transformed first fluorescence signal data from the composite fluorescence signal data) to generate the fluorescence signal data for the EF probe type (e.g., the “given probe type” as discussed herein).

Graph 506 of FIG. 5A shows the resolved fluorescence signal determined by subtracting the signal of graph 504 (i.e., the transformed first fluorescence signal) from the signal of graph 502 (i.e., the composite fluorescence signal). This signal is expected to estimate the fluorescence generated by the EF labels, separate from fluorescence attributable to the TaqMan probe labels. This signal may thus be used to quantify/analyze a target associated with the EFs in the reaction mixture (e.g., one or more target nucleic acids). The first fluorescence data or the transformed fluorescence signal data may be used to quantify/analyze a different target associated with the TaqMan probes in the reaction mixture.

Accordingly, the disclosed techniques may enable multiple targets/analytes to be advantageously assayed within the same reaction mixture through the same amplification process using probes with detectable labels (e.g., fluorophores) having spectral similarity, and, for example, detectable within the same detection channel (e.g., within the same fluorescence channel), thereby beneficially enabling increases in the plexy of multiplex assays without relying on additional dyes.

The graphs of FIG. 5B represent the same signal measurement types as in FIG. 5A, but in the assays producing the data of FIG. 5B, the EF probes were labeled with FAM dye and the TaqMan probes were labeled with VIC dye. Graph 508 shows the fluorescence signal generated by the EF labels (FAM), and graph 510 shows the fluorescence signal generated by the TaqMan probe labels (VIC). Graph 512 shows the insignificant change of fluorescence generated by the EF labels at the denaturation temperature (i.e., the EF label fluorescence remains substantially at its baseline signal level), whereas graph 514 shows the fluorescent signal generated by the TaqMan probe labels, which is substantially equal (after application of the linear function) to the signal measured at the annealing/extension temperature. Because the TaqMan and EF probes were differentially labelled in this assay, graph 516 shows a resolved signal for the EF label that essentially matches the EF signal at the annealing/extension temperature (in graph 508), and graph 518 shows the expected baseline signal for the TaqMan probe resulting from subtraction of the TaqMan label signal during denaturation from the TaqMan label signal during annealing/extension. The EF-associated fluorescence signal of graph 516 therefore closely represented a direct measurement of the signal from the EF labels (e.g., of graph 508).

FIG. 5C compares the derived EF-associated fluorescence signal 520 (graph 506 of FIG. 5A) as resolved from the assay of FIG. 5A with the measured EF-associated fluorescence signal 522 (graph 516 of FIG. 5B) which represents a direct measurement of EF label fluorescence. The results showed close correlation between the resolved and measured signals. The results therefore showed that fluorescent signals attributable to different probe types using the same label within the same detection channel can be separately resolved.

For further reference, FIG. 5D illustrates the fluorescence signal over cycle number measured during an annealing/extension stage (at a 65° C. temperature condition in this example) and during a denaturation stage (at a 95° C. temperature condition in this example) with TaqMan probe and EF probe compositions.

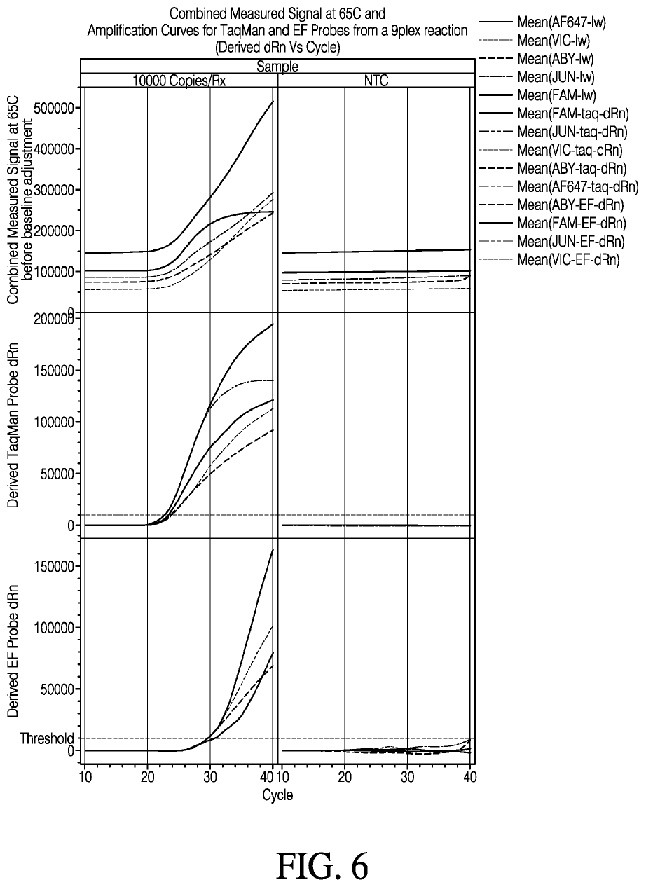

FIG. 6 illustrates the results of another assay test that included 5 different detection channels/dyes, four of which had a TaqMan probe and an EF probe with the same label (dye) in each channel and differing between the four channels. One of the channels had only the TaqMan probe. The results show that the fluorescent signals of the different probe types can be independently determined, and thus that a 9-plex reaction can be effectively carried out.

In view of the foregoing, in some implementations, disclosed embodiments may include obtaining different emission (e.g., fluorescence) signals at different temperatures, time periods (e.g., reaction stages of a thermal cycle), or other conditions of an amplification process (e.g., a PCR process) that involves multiple different probe types. The different probe types may comprise differential emission signal (e.g., fluorescence) responses at the different temperatures or time periods. The differential emission signal responses of the different probe types at the different temperatures, time periods, or other conditions may be used to determine isolated emission signals for the different probe types. This can therefore enable multiplex PCR assays that can quantify different target analytes utilizing emission signals that emit the same or substantially overlapping emission spectra (are spectrally similar), and thus, for example, can utilize a common detection channel for detecting multiple analytes.

### End-point PCR Embodiments

The analytical techniques in accordance with various embodiments can also be used when conducting a traditional end-point PCR process, in which the sample is subject to PCR in bulk (or larger reaction volumes not intended to capture a single or no DNA molecules using Poisson statistics), and as those of ordinary skill in the art are familiar with. In such a PCR process, the measurements of signal from the two different probe types can occur at an end-point cycle of PCR and at different reaction conditions (such as, e.g., denaturation and annealing and/or extension conditions as described herein), similar to the approaches described above. In the case of an end-point PCR process, the label signals detected will follow that outlined in FIG. 2A, with the signals thus indicating the presence or absence of the respective first and second targets. With reference to FIG. 3B, the measured end-point cycle signals under the two different reaction conditions thus may result in the differing levels of signal shown schematically at Cycle N+2 (analogizing that to the end-point cycle). By comparing the different signal levels obtained, using the algorithms set forth in FIG. 2A and further described above, the presence or absence of the first and second target nucleic acids can be determined using detectable labels having spectral similarity, for example, that can be detectable in a same detection channel.

Accordingly, a method for determining the presence or absence of multiple targets using the multiplexing techniques described herein can also be performed using endpoint PCR (i.e., using bulk or larger reaction volumes that are not of a size that is intended to rely on Poisson statistics and capturing a single molecule of target nucleic acid in the reaction volume as those of ordinary skill in the art are familiar with). The method employed in an end-point PCR application includes: preparing a reaction mixture comprising a first probe type (e.g., TaqMan probe) and a second probe type (e.g., an EF probe), configured to specifically interact with respective first and second nucleic acid targets; subjecting the reaction mixture to an amplification reaction (e.g., PCR); measuring end-point cycle signals of the reaction mixture at a first set of reaction conditions (e.g., denaturation conditions such as about 95° C.); and measuring an end-point signal of the reaction volumes at a second set of reaction conditions (e.g., annealing/extension conditions such as about 65° C.); determining a presence or absence the first and/or second nucleic acid targets in the reaction mixture by measuring during the first set of reaction conditions a first total emission (e.g., fluorescence) signal that comprises any first emission (e.g., fluorescence) signal if present, measuring during the second set of reaction conditions a second total emission (e.g., fluorescence) signal comprising any first emission (e.g., fluorescence) signal if present and any second emission (e.g., fluorescence) signal if present, and estimating the first emission (e.g., fluorescence) signal and/or second emission (e.g., fluorescence) signal based on the first and second total emission (e.g., fluorescence) signals. In other words, the techniques described above with reference to FIGS. 5A and 5B can be applied in an end-point PCR assay and analysis process.

### Extendable Fluorogenic (EF) Probe Template Formation

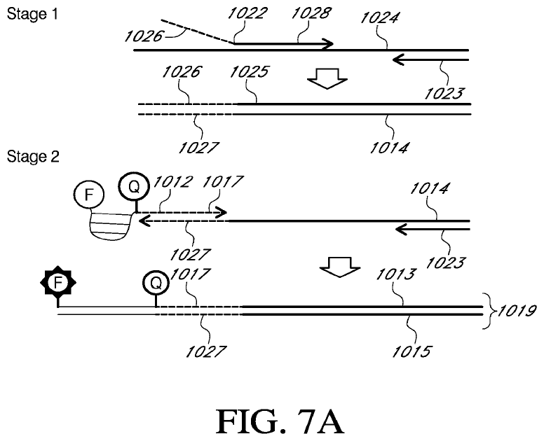

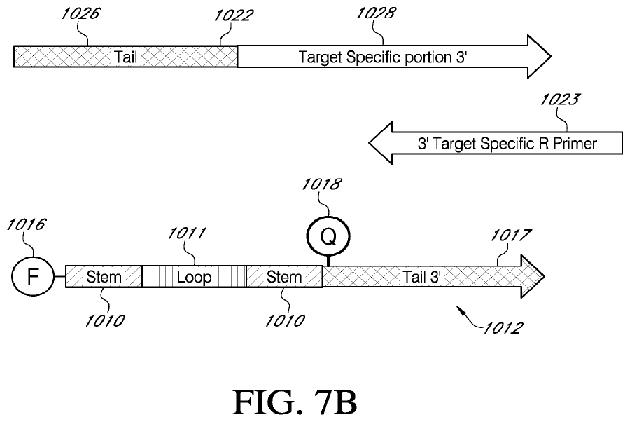

FIG. 7A illustrates a process of using a primer with a tail 1022, which is specific to a nucleic acid target 1024, to form the target template 1014 to which the EF probe 1012 can hybridize. The primer with a tail 1022 includes a tail 1026 and a target-specific portion 1028. FIG. 7B illustrates an example of the primer with a tail 1022 as a forward primer, a target specific primer 1023 paired with the primer with a tail 1022 as a reverse primer, and a more detailed view of the EF probe 1012.

As shown, in a first stage, the target-specific portion 1028 hybridizes to the target 1024. Extension of the target-specific portion 1028 forms a tailed amplicon 1025. Primer 1023, which is paired with the primer with a tail 1022, enables extension of the complement of the tailed amplicon 1025. It is this complement that forms the target template 1014. As shown, the target template 1014 includes a tail complement portion 1027.

In a second stage, the EF probe 1012 hybridizes to the target template 1014 and amplification can continue, for example, as shown in FIG. 3A. As shown, the EF probe 1012 includes a probe tail 1017 that has substantial homology with the tail 1026 and is therefore complementary to the tail complement portion 1027 of the target template 1014. Extension of probe 1012 and target template 1014 forms a double-stranded amplicon 1019. The primer 1023, shown here paired with the primer with a tail 122, may also function as the primer 1023 that pairs with the EF probe 11012 to enable formation of the double-stranded amplicon 1019, as shown in FIG. 3A.

As shown in FIG. 7B, the tail 1026 can form the 5′ end of the primer with a tail 1022. The EF probe 1012 can include a stem-loop portion, with stem portions 1010 on either side of a loop portion 1011, configured to form a stem-loop structure when the EF probe 1012 is single-stranded. For example, the label 1016 may be located on one side of the stem-loop portion and the quencher 1018 may be located on the opposite side of the stem-loop portion such that the label 1016 and quencher 1018 are brought into proximity when the stem-loop structure is formed but spaced farther apart when the EF probe 1012 is constrained into a more linear configuration (e.g., when incorporated into a double-stranded amplicon).

In the illustrated embodiment, the label 1016 is located at or near the 5′ end of the EF probe 1012 and the quencher 1018 is located 3′ of the label 1016. The positions of the label 1016 and quencher 1018 may be reversed in other embodiments. Preferably, as shown, the stem-loop portion is disposed 5′ of the probe tail 1017 so that the stem-loop portion remains at the end of the amplicons resulting from extension of the EF probe 1012, so that stem-loop structure formation (when single-stranded) is less likely to be compromised.

In addition to or alternative to the EF probes described herein, some embodiments may include other labelled oligonucleotides that generate increased fluorescence upon being incorporated into a double-stranded amplicon (relative to when in a single-stranded state), such as, for example during extension and/or annealing stages of a PCR process. For example, LUX™ primers include an internal fluorophore that is quenched by a hairpin structure located 5′ of the fluorophore. As with EF probes, a LUX™ primer provides increased fluorescence when incorporated into a double-stranded amplicon and the hairpin structure is linearized. Further, any of the primers or probes described herein may include one or more locked nucleic acids (LNAs) as are known in the art.

In some embodiments, the primer with a tail 1022 and the corresponding (non-tailed) primer 1023 are provided at different concentrations. For example, the primer 1023 may be provided at a higher concentration than the primer with a tail 1022. For example, the primer 1023 may be provided at a concentration that is about 2× (2 times) to about 30× the concentration of the primer with a tail 1022, or about 5× to about 25× the concentration of the primer with a tail 1022, or about 10× to about 20× the concentration of the primer with a tail 1022. Because the primer 1023 can function to both (1) drive the formation of the target template 1014 (as shown in FIG. 7A) and (2) drive the formation of the complement 1015 of the extended probe amplicon 1013 (as shown in FIG. 3A), providing it at a higher concentration than the corresponding primer with a tail 1022 can beneficially balance the reaction and help drive overall reaction efficiency.

In some embodiments, the EF probe 1012 is provided at a concentration that is different from the concentration of the primer with a tail 1022 and/or the concentration of the primer 1023. For example, the EF probe 1012 may be provided at a concentration that is greater than the concentration of the primer with a tail 1022 and that is less than the concentration of primer 1023. In some embodiments, the EF probe 1012 is provided at a concentration that is about 2× to about 20× the concentration of the primer with a tail 1022, or about 3× to about 15× the concentration of the primer with a tail 1022. As discussed above, providing the primer 1023 at a relatively higher concentration helps to drive the overall efficiency of the reaction. Providing the EF probe 1012 at a concentration that is higher than the primer with a tail 1022, but not necessarily higher than the primer 1023, pushes more of the associated amplification toward the EF probe 1012 as opposed to the primer with a tail 1022, yet still allows the primer 1023 to function as the primary driver of reaction efficiency.

In addition to or alternative to the “universal” EF probes that use a probe tail 1017, other embodiments include and/or utilize EF probes with a target-specific portion rather than a probe tail 1017. Such EF probes can directly hybridize to a target template nucleic acid as shown in FIG. 7A for generating a target template 114 with a tail complement portion 1027. In such embodiments, the probe tail 1017 of the EF probe 1012 is replaced with a target-specific portion that directly hybridizes to the target 1024. The process is otherwise similar to that shown in FIG. 3A. That is, after the EF probe is extended, a subsequent round of annealing/extension will extend the complement strand, forming the double-stranded amplicon that separates the fluorophore and the quencher to allow for fluorescence signal generation.

### Additional Label/Dye Details

Exemplary nonlimiting detectable labels that may be utilized with the embodiments described herein include, for example:
Fluoresceins (e.g., 5-carboxy-2,7-dichlorofluorescein, 5-Carboxyfluorescein (5-FAM), 6-JOE, 6-carboxyfluorescein (6-FAM), VIC, FITC, 6-carboxy-4′,5′-dichloro-2′,7′-dimethoxy-fluorescein (JOE)), 5 and 6-carboxy-1,4-dichloro-2′,7′-dichloro-fluorescein (TET), 5 and 6-carboxy-1,4-dichloro-2′,4′,5′,7′-tetra-chlorofluorescein, HEX, PET, NED, Oregon Green (e.g. 488, 500, 514));
Pyrenes; (e.g. Cascade Blue; Alexa Fluor 405);
Coumarins; (e.g. Pacific Blue, Atto 425, Alexa Fluor 350, Alexa Fluor 430);
Cyanine Dyes; (e.g. Cy dyes such as Cy3, Cy3.18, Cy3.5, Cy5, Cy5.18, Cy5.5, Cy7);
Rhodamines; (e.g., 110, 123, B, B 200, BB, BG, B extra, 5 and 6-carboxytetramethylrhodamine (5-TAMRA, 6-TAMRA), 5 and 6-Carboxyrhodamine 6G, Lissamine, Lissamine Rhodamine B, Rhod-2, ROX (6-carboxy-X-rhodamine), 5 and 6-ROX (carboxy-X-rhodamine), Sulphorhodamine B can C, Sulphorhodamine G Extra, 5 and 6 TAMRA (6-carboxytetramethyl-rhodamine), (TRITC), ABY, JUN, LIZ, RAD, RXJ, Texas Red; and Texas Red-X);
Alexa Fluor fluorophores (which is a broad class including many dye types such as cyanines) (e.g., Alexa 350, 405, 430, 488, 500, 514, 532, 546, 555, 568, 594, 610, 633, 635, 647, 660, 676, 680, 700, 750);

FRET donor/acceptor pairs (e.g., fluorescein/fluorescein, fluorescein/rhodamine, fluorescein/cyanine, rhodamine/cyanine, fluorescein/Alexa Fluor, Alexa Fluor/rhodamine); and other types of dyes known to those of skill in the art.

Fluorophore labels may be associated with quenchers such as dark fluorescent quencher (DFQ), black hole quenchers (BHQ), Iowa Black, QSY7, QSY21 quencher, Dabsyl and Dabcel sulfonate/carboxylate quenchers, and MGB-NFQ quenchers. Fluorophore labels may also include sulfonate derivatives of fluorescein dyes with SO3 instead of the carboxylate group, phosphoramidite forms of fluorescein, and/or phosphoramidite forms of Cy5, for example.

### Additional Amplification Details

Amplified products resulting from use of one or more embodiments described herein can be generated, detected, and/or analyzed on any suitable platform. In some embodiments, the nucleic acid targets may be single-stranded, double-stranded, or any other nucleic acid molecule of any size or conformation. The amplification processes described herein can include PCR (see, e.g., U.S. Pat. No. 4,683,202). In various embodiments, the PCR process can be real time or quantitative PCR (qPCR), end point PCR, digital PCR (dPCR), or any combination thereof.

In some embodiments, the amplification process includes reverse transcriptase PCR (RT-PCR). A disclosed method may include, for example, subjecting the target nucleic acid to a reverse transcription reaction prior to amplification via PCR. In some embodiments, the amplification process includes one-step RT-PCR (e.g., in a single vessel or reaction volume) in which one or more reverse transcriptases are used in combination with one or more DNA polymerases.

Optionally, certain qPCR assays can be plated into individual wells of a single array or multi-well plate, such as for example a TaqMan Array Card (see, e.g., Thermo Fisher Scientific, Waltham, MA; Catalog Nos. 4346800 and 4342265) or a MicroAmp multi-well (e.g., 96-well, 384-well) reaction plate (see, e.g., Thermo Fisher Scientific, Waltham, MA; Catalog Nos. 4346906, 4366932, 4306737, 4326659 and N8010560). Optionally, the different qPCR assays present in different wells of an array or plate can be dried or freeze-dried in situ and the array or plate can be stored or shipped prior to use. In some embodiments, the concepts described herein may be utilized in in situ hybridization applications not necessarily associated with PCR.

Other amplification methods, such as, e.g., loop-mediated isothermal amplification (“LAMP”), and other isothermal methods are also contemplated for use with the assay embodiments described herein.

The components described herein for enabling multiplexing utilizing probes with detectable labels having spectral similarity may be provided in a kit along with one or more additional components to enable an amplification process. Such components can include, for example, dNTPs, DNA polymerase, amplification buffers/reagents, master mix components as known in the art, and other components known in the art for enabling or assisting nucleic acid amplification.

### Additional Implementation Details

The principles disclosed herein may be implemented in various formats. For example, the various techniques discussed herein may be performed as a method that includes various acts for achieving particular results or benefits (e.g., the actions of flow diagram 400 of FIG. 4). In addition to the various techniques described above wherein data is collected at multiple time points during various cycles of an amplification process (e.g., a qPCR assay), data can be collected at an end-point cycle of an amplification process (e.g., an endpoint PCR assay) and analyzed in accordance with the techniques described herein. Such an endpoint data collection technique may be employed, for example, in genotyping applications. The various techniques for data collection and analysis disclosed are not mutually exclusive and can be utilized in combination. In some instances, the techniques discussed herein are represented in computer-executable instructions that may be stored on one or more hardware storage devices. The computer-executable instructions may be executable by one or more processors to carry out (or to configure a system to carry out) the disclosed techniques. In some embodiments, a system may be configured to send the computer-executable instructions to a remote device to configure the remote device for carrying out the disclosed techniques.

The various specific temperatures, times, and rates of temperature change depicted in the figures and discussed with respect to various embodiments are illustrative only and should not be understood as limiting of the scope of the present disclosure and claims. Other specific temperatures, times, and rates of temperature change can be employed and will be understood from the remainder of the disclosure. Moreover, the different sets of conditions under which detection of emission signal occurs can vary from each other in different cycles. For example, a denaturation temperature may vary over different cycles, but still be considered a common set of conditions for taking measurements. Likewise, with the conditions of various annealing and/or extension stages in different cycles.

Systems for implementing the disclosed embodiments may include various components, such as, by way of non-limiting example, processor(s), storage, sensor(s), I/O system(s), communication system(s), etc.

The processor(s) may comprise one or more sets of electronic circuitries that include any number of logic units, registers, and/or control units to facilitate the execution of computer-readable instructions (e.g., instructions that form a computer program). Such computer-readable instructions may be stored within storage. The storage may comprise physical system memory and may be volatile, non-volatile, or some combination thereof. Furthermore, storage may comprise local storage, remote storage (e.g., accessible via communication system(s) or otherwise), or some combination thereof.

In some implementations, the processor(s) may comprise or be configurable to execute any combination of software and/or hardware components that are operable to facilitate processing using machine learning models or other artificial intelligence-based structures/architectures. For example, processor(s) may comprise and/or utilize hardware components or computer-executable instructions operable to carry out function blocks and/or processing layers configured in the form of, by way of non-limiting example, single-layer neural networks, feed forward neural networks, radial basis function networks, deep feed-forward networks, recurrent neural networks, long-short term memory (LSTM) networks, gated recurrent units, autoencoder neural networks, variational autoencoders, denoising autoencoders, sparse autoencoders, Markov chains, Hopfield neural networks, Boltzmann machine networks, restricted Boltzmann machine networks, deep belief networks, deep convolutional networks (or convolutional neural networks), deconvolutional neural networks, deep convolutional inverse graphics networks, generative adversarial networks, liquid state machines, extreme learning machines, echo state networks, deep residual networks, Kohonen networks, support vector machines, neural Turing machines, and/or others.

In some instances, actions performable by a system may rely at least in part on communication system(s) for receiving information from remote system(s), which may include, for example, separate systems or computing devices, sensors, and/or others. The communications system(s) may comprise any combination of software or hardware components that are operable to facilitate communication between on-system components/devices and/or with off-system components/devices. For example, the communications system(s) may comprise ports, buses, or other physical connection apparatuses for communicating with other devices/components. Additionally, or alternatively, the communications system(s) may comprise systems/components operable to communicate wirelessly with external systems and/or devices through any suitable communication channel(s), such as, by way of non-limiting example, Bluetooth, ultra-wideband, WLAN, infrared communication, and/or others.

A system may comprise or be in communication with sensor(s). Sensor(s) may comprise any device for capturing or measuring data representative of perceivable or detectable phenomena. By way of non-limiting example, the sensor(s) may comprise one or more light sensors/detectors, microphones, thermometers, barometers, magnetometers, accelerometers, gyroscopes, and/or others.

Furthermore, a system may comprise or be in communication with I/O system(s). I/O system(s) may include any type of input or output device such as, by way of non-limiting example, a touch screen, a mouse, a keyboard, a controller, a speaker and/or others, without limitation. For example, the I/O system(s) may include a display system that may comprise any number of display panels, optics, laser scanning display assemblies, and/or other components. One will appreciate, in view of the present disclosure, that the sensor(s) may, in some instances, be utilized as I/O system(s).

Disclosed embodiments may comprise or utilize a special purpose or general-purpose computer including computer hardware, as discussed in greater detail below. Disclosed embodiments also include physical and other computer-readable media for carrying or storing computer-executable instructions and/or data structures. Such computer-readable media can be any available media that can be accessed by a general-purpose or special-purpose computer system. Computer-readable media that store computer-executable instructions in the form of data are one or more “physical computer storage media” or “hardware storage device(s).” Computer-readable media that merely carry computer-executable instructions without storing the computer-executable instructions are “transmission media.” Thus, by way of example and not limitation, the current embodiments can comprise at least two distinctly different kinds of computer-readable media: computer storage media and transmission media.

Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in hardware in the form of computer-executable instructions, data, or data structures and that can be accessed by a general-purpose or special-purpose computer.

A “network” may comprise one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmission media can include a network and/or data links which can be used to carry program code in the form of computer-executable instructions or data structures and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special purpose computer, or special purpose processing device to perform a certain function or group of functions. The computer-executable instructions may be, for example, binaries, intermediate format instructions such as assembly language, or even source code.

Disclosed embodiments may comprise or utilize cloud computing. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Those skilled in the art will appreciate that the invention may be practiced in network computing environments with many types of computer system configurations. including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, wearable devices, and the like. The invention may also be practiced in distributed system environments where multiple computer systems (e.g., local and remote systems), which are linked through a network (either by hardwired data links, wireless data links, or by a combination of hardwired and wireless data links), perform tasks. In a distributed system environment, program modules may be located in local and/or remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Application-specific Standard Products (ASSPs), System-on-a-chip systems (SOCs), Complex Programmable Logic Devices (CPLDs), central processing units (CPUs), graphics processing units (GPUs), and/or others.

As used herein, the terms “executable module,” “executable component,” “component,” “module,” or “engine” can refer to hardware processing units or to software objects, routines, or methods that may be executed on one or more computer systems. The different components, modules, engines, and services described herein may be implemented as objects or processors that execute on one or more computer systems (e.g., as separate threads).

In various implementations, a system for analyte detection in a multiplexed polymerase chain reaction (PCR) process and include one or more processors of at least one computing device; and a memory storing one or more instructions which, when executed by the one or more processors, cause the one or more processors to perform a process of: obtaining, at one or more time points during one or more cycles of said PCR process, measured emission signal data associated with a composite emission signal from at least a first probe type that can include a first label configured to generate a first emission signal and a second probe type that can include a second label configured to generate a second emission signal which has spectrally similar characteristics as said first emission signal, said first probe type and said second probe type differing in thermal and/or temporal properties; and determining, based at least partially on said emission signal data associated with said composite emission signal and thermal and/or temporal properties of one or more of said first probe type and said second probe type, resolved emission signal data associated with respective emission signal from a given probe type of one or more of said first probe type and said second probe type during said one or more cycles of said PCR process.

Additional implementations of the system can also include one or more of the following features in any combination. The first label is the same as said second label. The first and second labels are first and second fluorophores, respectively, and the first and second emission signals are first and second fluorescence signals, respectively. The obtaining occurs during a first set of reaction conditions and during a second set of reaction conditions of an end-point cycle of said PCR process, where the first set of reaction conditions and the second set of reaction conditions differ from each other. The amplification process is an end-point PCR process. The first probe type has binding affinity to a first analyte and the second probe type has binding affinity to a second analyte different from said first analyte. The determining can be performed in real-time during said one or more cycles of said PCR process. The obtaining and determining can be executed by different processors. The first probe type is cleavable and the second probe type is non-cleavable. The first probe type can include a Taqman probe, and the second probe type can include an extendable probe. The non-cleavable probe type can include an extendable fluorogenic probe. The instructions which, when executed by the one or more processors, further cause the one or more processors to perform a process of: obtaining additional measured emission signal data during one or more cycles of the PCR process associated with first temperature condition and/or time period of the one or more cycles, and where the measured emission signal data associated with the composite emission signal are associated with second temperature condition and/or time period of the one or more cycles, the second temperature condition and/or time period differing from the first temperature condition and/or time period. The determining can utilize the measured emission signal data associated with the composite emission signal and the additional measured emission signal data as inputs for generating the resolved emission signal data associated with the respective emission signal from the given probe type. Utilizing the measured emission signal data associated with the composite emission signal and the additional measured emission signal data as inputs for generating the resolved emission signal data associated with the respective emission signal from the given probe type can include: generating transformed emission signal data by applying a transformation to the additional emission signal data; and modifying the measured emission signal data associated with the composite emission signal with the transformed emission signal data to generate the resolved emission signal data associated with the respective emission signal from the given probe type. The transformed emission signal data can indicate an approximate emission associated with the first probe type at the second temperature condition and/or time period.

The one or more instructions of the system, when executed by the one or more processors, can further cause the one or more processors to perform a process of: quantifying a first target associated with the first probe type based upon at least the additional measured emission signal data; and quantifying a second target associated with the second probe type based upon at least the resolved emission signal data associated with the respective emission signal from the given probe type. The quantifying the first target can include determining a concentration of the first target in a sample subjected to the PCR process and the quantifying the second target can include determining a concentration of the second target in a sample subjected to the PCR process. The first temperature condition and/or time period does not overlap with the second temperature condition and/or time period. The first temperature condition is higher than the second temperature condition. The second temperature condition is in a range from about 45 to about 75 c. The first temperature condition is in a range from about 80 to about 100 c. The second temperature and/or time condition is associated with an annealing stage, an extension stage, or a combined annealing and extension stage of the PCR process. The first temperature condition and/or time period is associated with a denaturing stage of the PCR process. The PCR process can include a plurality of PCR cycles. The first probe type can be associated with a cumulative emission signal that stays substantially stable across multiple stages of a PCR cycle and increases cumulatively over multiple PCR cycles. The second probe type can be associated with a transient emission signal that fluctuates over multiple stages in each PCR cycle. Implementations of the described techniques can include hardware, a method or process, or computer software on a computer-accessible medium.

In various implementations, a method for enabling an analyte detection in a multiplexed polymerase chain reaction (PCR) process can include obtaining, at one or more time points during one or more cycles of said PCR process, measured emission signal data associated with a composite emission signal from at least a first probe type can include a first label configured to emit a first emission signal and a second probe type can include a second label configured to emit a second emission signal which has spectrally similar characteristics as said first emission signal, said first probe type and said second probe type differing in thermal and/or temporal properties; and determining, based at least partially on said emission signal data associated with said composite emission signal and thermal and/or temporal properties of one or more of said first probe type and said second probe type, resolved emission signal data associated with respective emission signal from a given probe type of said first probe type and said second probe type during said one or more cycles of said PCR process.

Additional implementations of such a method can also include one or more of the following features, in any combination. The first label can be the same as said second label. The first and second labels can be first and second fluorophores, respectively, and the first and second emission signals can be first and second fluorescence signals, respectively. The obtaining can occur during a first set of reaction conditions and during a second set of reaction conditions of an end-point cycle of said PCR process, where the first set of reaction conditions and the second set of reaction conditions differ from each other. The amplification process can be an end-point PCR process. The first probe type can have binding affinity to a first analyte and said second probe type can have binding affinity to a second analyte different from said first analyte. The first probe type can be cleavable and the second probe type can be non-cleavable. The first probe type can include a Taqman probe, and the second probe type can include an extendable fluorogenic probe. The non-cleavable probe type can be an extendable fluorogenic probe.

The method can further include obtaining additional measured emission signal data during one or more cycles of the PCR process associated with first temperature condition and/or time period of the one or more cycles, and where the measured emission signal data associated with the composite emission signal are associated with second temperature condition and/or time period of the one or more cycles, the second temperature condition and/or time period differing from the first temperature condition and/or time period. The determining can utilize the measured emission signal data associated with the composite emission signal and the additional measured emission signal data as inputs for generating the resolved emission signal data associated with the respective emission signal from the given probe type. Utilizing the measured emission signal data associated with the composite emission signal and the additional measured emission signal data as inputs for generating the resolved emission signal data associated with the respective emission signal from the given probe type can include: generating transformed emission signal data by applying a transformation to the additional emission signal data; and modifying the measured emission signal data associated with the composite emission signal with the transformed emission signal data to generate the resolved emission signal data associated with the respective emission signal from the given probe type. The transformed emission signal data can indicate an approximate emission associated with the first probe type at the second temperature condition and/or time period.

The method can also include any of the actions that are described as being executable by the instructions above.

In yet other implementations, a computer-readable media can store one or more instructions which that can be further implementable with one or more of the combinations of features as set forth above for the system and/or to perform the actions of the method implementations.

One will also appreciate how any feature or operation disclosed herein may be combined with any one or combination of the other features and operations disclosed herein. Additionally, the content or feature in any one of the figures may be combined or used in connection with any content or feature used in any of the other figures. In this regard, the content disclosed in any one figure is not mutually exclusive and instead may be combinable with the content from any of the other figures.

The described embodiments are to be considered in all respects only as illustrative and not restrictive. All changes which come within the meaning and range of equivalency of the claims are to be embraced within their scope.

What is claimed is:

1. A system for analyte detection in a multiplexed polymerase chain reaction (PCR) process, comprising:
one or more processors of at least one computing device; and
a memory storing one or more instructions which, when executed by the one or more processors, cause the one or more processors to perform a process of:
obtaining, at one or more time points during one or more cycles of said PCR process, measured emission signal data associated with a composite emission signal from at least a first probe type comprising a first label configured to generate a first emission signal and a second probe type comprising a second label configured to generate a second emission signal which has spectrally similar characteristics as said first emission signal, said first probe type and said second probe type differing in thermal and/or temporal properties; and
determining, based at least partially on said emission signal data associated with said composite emission signal and thermal and/or temporal properties of one or more of said first probe type and said second probe type, resolved emission signal data associated with respective emission signal from a given probe type of one or more of said first probe type and said second probe type during said one or more cycles of said PCR process.

2. The system of claim 1, wherein said first label is the same as said second label.

3. The system of claim 1, wherein said first probe type has binding affinity to a first analyte and said second probe type has binding affinity to a second analyte different from said first analyte.

4. The system of claim 1, wherein said determining is performed in real-time during said one or more cycles of said PCR process.

5. The system of claim 1, wherein said obtaining and determining are executed by different processors.

6. The system of claim 1, wherein the first probe type is cleavable and the second probe type is non-cleavable.

7. The system of claim 6, wherein the first probe type comprises a TaqMan probe, and the second probe type comprises an extendable probe.

8. The system of claim 6, wherein the non-cleavable probe type comprises an extendable fluorogenic probe.

9. The system of claim 1, wherein the instructions which, when executed by the one or more processors, further cause the one or more processors to perform a process of:
obtaining additional measured emission signal data during one or more cycles of the PCR process associated with first temperature condition and/or time period of the one or more cycles, and
wherein the measured emission signal data associated with the composite emission signal are associated with second temperature condition and/or time period of the one or more cycles, the second temperature condition and/or time period differing from the first temperature condition and/or time period.

10. The system of claim 9, wherein said determining utilizes the measured emission signal data associated with the composite emission signal and the additional measured emission signal data as inputs for generating the resolved emission signal data associated with the respective emission signal from the given probe type.

11. The system of claim 9, wherein utilizing the measured emission signal data associated with the composite emission signal and the additional measured emission signal data as inputs for generating the resolved emission signal data associated with the respective emission signal from the given probe type comprises:
generating transformed emission signal data by applying a transformation to the additional emission signal data; and
modifying the measured emission signal data associated with the composite emission signal with the transformed emission signal data to generate the resolved emission signal data associated with the respective emission signal from the given probe type.

12. The system of claim 11, wherein the transformed emission signal data indicates an approximate emission associated with the first probe type at the second temperature condition and/or time period.

13. The system of claim 11, wherein the one or more instructions, when executed by the one or more processors, further cause the one or more processors to perform a process of:
quantifying a first target associated with the first probe type based upon at least the additional measured emission signal data; and
quantifying a second target associated with the second probe type based upon at least the resolved emission signal data associated with the respective emission signal from the given probe type.

14. The system of claim 13, wherein the quantifying the first target comprises determining a concentration of the first target in a sample subjected to the PCR process and the quantifying the second target comprises determining a concentration of the second target in a sample subjected to the PCR process.

15. The system of claim 9, wherein the first temperature condition and/or time period does not overlap with the second temperature condition and/or time period.

16. The system of claim 9, wherein the first temperature condition is higher than the second temperature condition.

17. The system of claim 9, wherein the second temperature condition is in a range from about 45° to about 75° C.

18. The system of claim 9, wherein the first temperature condition is in a range from about 80° to about 100° C.

19. The system of claim 9, wherein the second temperature and/or time condition is associated with an annealing stage, an extension stage, or a combined annealing and extension stage of the PCR process.

20. The system of claim 9, wherein the first temperature condition and/or time period is associated with a denaturing stage of the PCR process.

21. The system of claim 9, wherein the PCR process comprises a plurality of PCR cycles.

22. The system of claim 21, wherein the first probe type is associated with a cumulative emission signal that stays substantially stable across multiple stages of a PCR cycle and increases cumulatively over multiple PCR cycles.

23. The system of claim 21, wherein the second probe type is associated with a transient emission signal that fluctuates over multiple stages in each PCR cycle.

24. The system of claim 1, wherein the first and second labels are first and second fluorophores, respectively, and the first and second emission signals are first and second fluorescence signals, respectively.

25. The system of claim 1, wherein the obtaining occurs during a first set of reaction conditions and during a second set of reaction conditions of an end-point cycle of said PCR process, wherein the first set of reaction conditions and the second set of reaction conditions differ from each other.

26. The system of claim 1, wherein the amplification process is an end-point PCR process.

27. A method for enabling an analyte detection in a multiplexed polymerase chain reaction (PCR) process, comprising:
obtaining, at one or more time points during one or more cycles of said PCR process, measured emission signal data associated with a composite emission signal from at least a first probe type comprising a first label configured to emit a first emission signal and a second probe type comprising a second label configured to emit a second emission signal which has spectrally similar characteristics as said first emission signal, said first probe type and said second probe type differing in thermal and/or temporal properties; and
determining, based at least partially on said emission signal data associated with said composite emission signal and thermal and/or temporal properties of one or more of said first probe type and said second probe type, resolved emission signal data associated with respective emission signal from a given probe type of said first probe type and said second probe type during said one or more cycles of said PCR process.

28-50. (canceled)

51. Computer-readable media storing one or more instructions which, when executed by one or more processors of at least one computing device, cause the one or more processors to perform a process of:
obtaining, at one or more time points during one or more cycles of a PCR process, measured emission signal data associated with a composite emission signal from at least a first probe type comprising a first label configured to generate a first emission signal and a second probe type comprising a second label configured to generate a second emission signal which has spectrally similar characteristics as said first emission signal, said first probe type and said second probe type differing in thermal and/or temporal properties; and
determining, based at least partially on said emission signal data associated with said composite emission signal and thermal and/or temporal properties of one or more of said at least said first probe type and said second probe type, resolved emission signal data associated with respective emission signal from a given probe type of said at least said first probe type and said second probe type during said one or more cycles of said PCR process.

52-74. (canceled)
